(19) United States
(12) Patent Application Publication
(10) Pub. No.: US 2023/0119571 A1
(43) Pub. Date: Apr. 20, 2023

(54) GUIDE SUPPORT FOR DELIVERING A MEDICAL DEVICE

(71) Applicant: SKW Cardiovascular Technologies, Escondido, CA (US)

(72) Inventor: Subeer Wadia, Escondido, CA (US)

(73) Assignee: SKW Cardiovascular Technologies, Escondido, CA (US)

(21) Appl. No.: 18/047,631

(22) Filed: Oct. 18, 2022

Related U.S. Application Data
(60) Provisional application No. 63/257,112, filed on Oct. 18, 2021.

(51) Int. Cl. A61M 25/10

(52) U.S. Cl. CPC A61M 25/1034; A61M 25/104; A61M 25/10181; A61M 2025/1063; A61M 2025/1079

(57) ABSTRACT

A guide extension catheter with improved stability includes an elongated pushing portion connected to an extension portion with a balloon disposed near its distal end. An inflation lumen extends along a length of the pushing portion and the extension portion to feed fluid into an interior of the balloon. Introduction of fluid into the balloon expands it radially outward from the extension to apply pressure against an inner wall of the vessel to anchor the distal end of the extension within the vessel.

### RELATED APPLICATIONS

This application claims the benefit of the priority of U.S. Application No. 63/257,112, filed Oct. 18, 2021, which is incorporated herein by reference in its entirety.

### FIELD OF THE INVENTION

The present invention relates generally to guide catheters for performing medical procedures within vasculature, and more specifically to a guide support for stabilizing guide extension catheters to facilitate delivery of treatment to hard-to-reach sites.

### BACKGROUND

Coronary artery disease (CAD) and other diseases of the peripheral vasculature are often treated by a balloon angioplasty and/or stent placement. The advancement of revascularization devices, e.g., balloons or stent delivery systems, within blood vessels to a treatment site is a challenge where deposits such as plaque and other build-ups in the vessels act as mechanically resistant obstructions to advancement of the devices to the treatment site.

In a procedure commonly referred to as Percutaneous Coronary Intervention (PCI), a stent is inserted to help to improve coronary artery blood flow and reduce chest pain. Stents have been proven to improve survivability in the event of an acute myocardial infarction. For placement within the vasculature, the stent is often compressed and mounted on a balloon catheter. The positioning of the stent on the catheter can reduce the flexibility of the balloon and resists its smooth advancement. This can make it difficult or impossible to deliver the stent to a treatment site, i.e., the lesion, and risks dislodgement of the un-deployed stent from its delivery balloon.

Guide catheter extensions can be inserted within a larger guide catheter to provide added support for the crossing of lesions or for the distal delivery of balloons and stents. This technique is used for deep seating the guide catheter within the ostium of the coronary artery. Guide catheter extension devices have become a mainstay for coronary intervention because of their ability to facilitate device delivery. Recent data suggests that approximately 18-20% of PCIs are now performed with guide extension. The large majority of these are performed as a “bailout” after stent delivery has failed using conventional guiding catheter support techniques. The need for guide catheter extension become more important as operators move to an increased use of radial interventions, with less guiding catheter backup support, and more complex PCI. In most cases, the guide extension is used to obtain additional backup by advancing the tip of the guide extension system into the proximal or midportion of a coronary artery. A number of guide catheter extension systems have been developed and are commercially available.

One example of a commercially available guide extension is the “Guideliner® (Teleflex Incorporated, Morrisville, N.C., USA), described in U.S. Pat. No. 8,292,850 of Root, et al. This device is a coaxial guide catheter to be passed through a lumen of a guide catheter for use with interventional cardiology devices that are insertable into a branch artery that branches off from a main artery. This coaxial guide catheter is extended through the lumen of the guide catheter and beyond its distal end for insertion into the branch artery. The guide extension is supported by a tapered inner catheter with an atraumatic tip to avoid vessel injury, while advancing the guide extension into the proximal portion of a coronary vessel, providing additional “backup” support for delivery of the stent or a balloon.

Another commercially available guide extension system is the “Guidezilla™” (Boston Scientific, Marlborough, Mass., USA), which is described in U.S. Pat. No. 9,764,118 of Anderson, et al. This guide extension system uses a push member having a proximal portion having a proximal stiffness, a distal portion having a distal stiffness different from the proximal stiffness, and a transition portion which provides a smooth transition between the proximal and distal portions. A distal tubular member is attached to the push member and has an outer diameter larger than the outer diameter of the push member.

The Guideliner, Guidezilla™, and another system, the Telescope™ (Medtronic, Santa Rosa, Calif.) are “first generation” guide catheter extension devices. They are monorail delivered, tubular structures, often requiring continued manipulation to achieve delivery through tortuous vessels. The TrapLiner® guide extension catheter (Teleflex Incorporated) modifies these designs by adding a trapping balloon near the distal end of the push rod to trap the guide wire against the interior wall of the guide catheter. While these guide extensions generally permit closer approach to the lesion and provide additional support in crossing the lesion to be treated, the distal end of the extension is unsupported, and the lesion can still be difficult to pass through to allow the guide wire and its cargo to be advanced. Further, while these techniques may be successful, they require additional time, radiation dosing, and contrast, and may cause balloon barotrauma in proximal, non-target segments of the coronary artery, with potential risks of dissection, or restenosis.

In view of the limitations of existing guide extensions, a device and method that would permit stabilization of the distal end of the tubular guide extension system to, or ideally, beyond, the lesion to be treated, would have significant advantages over existing guide extension devices. The present invention is directed to such a device.

### SUMMARY

According to embodiments of the inventive device, an improved guide catheter extension device facilitates intravascular procedures with enhanced delivery capability by employing a compliant balloon disposed on the outer surface near the distal end of the catheter extension. The annular balloon, the center portion of which can be expanded/deflated via an inflation lumen that extends along the length of the catheter extension, has thin edges on either side of the center that fit tightly to the outer surface of the extension. The outer surface of the balloon is configured to expand radially to directly contact and apply uniform pressure against the inner walls of the vessel. The expanded balloon acts to anchor the guide extension in place against the inner walls of the vasculature, thus centering and stabilizing the distal end of the extension. This allows the guide wire to be advanced and/or retracted smoothly relative to the end of the extension. This approach may be used with virtually any blood vessel, capillary, artery, arteriole, or branch vessel, including, but not limited to, coronary arteries, coronary arterioles, and coronary capillaries, peripheral arteries, peripheral veins, pulmonary arteries, pulmonary arterioles, and pulmonary capillaries.

Interventional cardiology procedures are typically carried out under fluoroscopy or another x-ray or imaging technique. In some embodiments, one or more radio-opaque marker may be disposed near the distal end of the extension. In one implementation, the marker may be formed by laminating the outer surface of the extension at a location that is then covered by the distal tail of the balloon.

The outer surface of the balloon may optionally be coated with, or the balloon material may be impregnated with, one or more substances to aid in the treatment or in the manipulation of the device. For example, a pharmacological agent, i.e., drug or medication, may be incorporated into the balloon surface for application at the site of the stenosis. Upon inflation, cells or micropores within the balloon's surface expand, releasing their payload to the surrounding vessel walls. The pharmacological agent may include an additive to enhance absorption of the drug into the vascular wall. In another embodiment, one or more drug or a combination of drug(s) and excipients may be contained within a polymer coating that is applied to the balloon to allow the drug to diffuse into the vessel wall when contacted by the expanded balloon. The body of the guide extension catheter may be coated with a friction-reducing material and/or a hydrophilic material to enhance maneuverability of the distal end of the guide extension. The stiffness of the guide extension catheter may be substantially uniform along its full length or there may be defined areas that have variable stiffness to modify flexibility of the assembly. In one example, the guide extension catheter may be more flexible near its distal end than at its proximal end.

Expansion of the balloon may be effected by injecting a fluid through the inflation lumen, which is connected at its proximal end to a Luer connector or similar connector. Non-limiting examples of appropriate fluids include iodinated contrast solutions, saline solutions, sterile water, and air. The use of a syringe allows injection of a precisely measured volume of fluid appropriate for the balloon size into the connector by applying gradual and constant force. Alternatively, a small pump may be used to inject and withdraw the fluid from the balloon.

Inflation of the balloon acts to resist movement of the guide extension catheter as the result of reactionary counterforces within the vessel, particularly when advancing equipment (such as balloons or stents) through stenotic or tortuous segments within the vessel. The inventive guide extension catheter assists in delivering at least one of microcatheters, stents, and coronary balloons. Once the balloon inflates and pushes against the inner surface of the blood vessels, it stabilizes the guide extension to facilitate advancing equipment such as microcatheters, stents, and coronary balloons to a target location.

During a procedure using the inventive device, the balloon may be inflated once the guide extension catheter abuts a stenotic lesion. The balloon may be inflated immediately upon the guide extension catheter arriving at the location of the stenotic lesion, as determined by imaging of the radio-opaque marker, or it can be inflated afterward, for example, once the practitioner has determined that the resistance presented by the lesion cannot be overcome with additional attempts to further advance the catheter. The balloon may also be inflated when the guide extension catheter can simply no longer be advanced due to narrowing in the blood vessel, apart from encountering a lesion. Once delivery of the treatment has been completed, the balloon is deflated for withdrawal of the catheter.

The guide extension catheter may be made in a number of sizes that fall within a range of 3 French and 30 French (3 FR-30 Fr). Such sizes are well known for use in a variety of diagnostic and interventional procedures. Selection of the appropriate size guide extension catheter to be used will depend on the size of the vessel to which treatment is to be delivered and will be apparent to a person of skill in the art.

In one aspect of the invention, a guide extension catheter with improved stability includes an elongated pushing portion configured for guiding a guide wire into a vessel, the pushing portion having a proximal end and a distal end; an extension portion disposed at the distal end of the pushing portion, the extension portion comprising a tube having a proximal portion, a distal portion, and an interior configured for slidably receiving a guide wire configured for delivery of a treatment device to a treatment target; a balloon disposed near a distal end of the extension portion, the balloon having an annular configuration with an expandable center portion and edges on either side of the center portion, the edges sealed to an outer surface of the extension portion to define a fluid-tight seal; an inflation lumen extending along a length of the pushing portion and the extension portion, wherein a distal end of the inflation lumen feeds into an interior of the balloon; and a fluid connecter connected to a proximal end of the inflation lumen, the fluid connector configured for introducing fluid into the inflation lumen to expand the balloon; wherein introduction of fluid into the balloon causes the balloon to expand radially outward from the extension to apply pressure against an inner wall of the vessel to anchor the distal end of the extension within the vessel. The elongated pushing portion may be a hypotube formed of a metal material, where the inflation lumen is retained within an interior of the hypotube. The exterior surface of the elongated pushing portion nay be at least partially coated with a low-friction material. The tube of the extension portion may be formed of a flexible polymer material having an inner surface coated with a low-friction material. At least a portion of a length of the tube may be reinforced with a coiled wire. In some embodiments, the tube may be formed of polymer materials having different stiffnesses, wherein the proximal portion of the extension has greater stiffness than the distal portion. The extension portion may further include a polymer jacket disposed on an outer surface of the tube, where the polymer jacket secures the inflation lumen against the outer surface of the tube. A radio-opaque marker may be formed on an outer surface of the tube, sandwiched beneath a distal-most edge of the balloon.

In another aspect of the invention, an improved guide extension catheter comprising an elongated pushing portion having a distal end connected to a tubular extension portion includes the improvement of a balloon disposed near a distal end of the extension portion, the balloon having an annular configuration with an expandable center portion and edges on either side of the center portion, the edges sealed to an outer surface of the extension portion to define a fluid-tight seal. An inflation lumen extends along a length of the pushing portion and the extension portion, where a distal end of the inflation lumen feeds into an interior of the balloon. A fluid connecter is connected to a proximal end of the inflation lumen, the fluid connector configured for introducing fluid into the inflation lumen to expand the balloon, so that introduction of fluid into the balloon causes the balloon to expand radially outward from the extension to apply pressure against an inner wall of the vessel to anchor the distal end of the extension within the vessel. The elongated pushing portion may be a hypotube formed of a metal material, where the inflation lumen is retained within an interior of the hypotube. The exterior surface of the elongated pushing portion nay be at least partially coated with a low-friction material. The tube of the extension portion may be formed of a flexible polymer material having an inner surface coated with a low-friction material. At least a portion of a length of the tube may be reinforced with a coiled wire. In some embodiments, the tube may be formed of polymer materials having different stiffnesses, wherein the proximal portion of the extension has greater stiffness than the distal portion. The extension portion may further include a polymer jacket disposed on an outer surface of the tube, where the polymer jacket secures the inflation lumen against the outer surface of the tube. A radio-opaque marker may be formed on an outer surface of the tube, sandwiched beneath a distal-most edge of the balloon.

In still another aspect of the invention, in improved method for delivering a treatment device to a target location within peripheral vasculature of a subject, where the method includes advancing a guide wire toward a target location within the peripheral vasculature using a guide catheter, the improvement involves providing a guide extension catheter having an elongated pushing portion having a distal end connected to a tubular extension portion configured for slidably receiving the guide wire, wherein the extension portion has a balloon disposed near a distal end of the extension portion, the balloon having an annular configuration with an expandable center portion with sealed edges on either side of the center portion defining a fluid-tight seal, and an inflation lumen extending along a length of the pushing portion and the extension portion, wherein a distal end of the inflation lumen feeds into an interior of the balloon; advancing the guide extension catheter and the guide wire through the guide catheter and into the subject's peripheral vasculature toward the target location until resistance is encountered; stabilizing a distal end of the guide extension catheter within the peripheral vasculature by injecting a fluid into a proximal end of the inflation lumen and into the balloon to expand the balloon to apply pressure against an inner surface of the peripheral vasculature; advancing a distal end of the guide wire beyond the distal end of the guide extension catheter to the target location; delivering the treatment device to the target location; retracting the fluid from the balloon; and withdrawing the guide extension catheter, guide wire, and guide catheter from the subject. The elongated pushing portion may be a hypotube formed of a metal material, where the inflation lumen is retained within an interior of the hypotube. The exterior surface of the elongated pushing portion nay be at least partially coated with a low-friction material. The tube of the extension portion may be formed of a flexible polymer material having an inner surface coated with a low-friction material. At least a portion of a length of the tube may be reinforced with a coiled wire. In some embodiments, the tube may be formed of polymer materials having different stiffnesses, wherein the proximal portion of the extension has greater stiffness than the distal portion. The extension portion may further include a polymer jacket disposed on an outer surface of the tube, where the polymer jacket secures the inflation lumen against the outer surface of the tube. A radio-opaque marker may be formed on an outer surface of the tube, sandwiched beneath a distal-most edge of the balloon. The treatment device may be at least one of a stent, a coronary balloon, and a microcatheter.

### BRIEF DESCRIPTION OF THE DRAWINGS

FIG. 1 is a schematic view of a guide catheter and a guide extension catheter located in the aortic arch and coronary.

FIG. 2A is a side plan view of a guide support according to an embodiment of the invention; FIG. 2B is a detain view of a section of the support of FIG. 2A.

FIGS. 3A-3C are views of an extension section according to an embodiment of the inventive guide support, where FIG. 3A is a side elevation of the distal end of the extension with the balloon inflated; FIG. 3B is a cross-sectional view taken along line A-A of FIG. 3A, and FIG. 3C is a side elevation of the distal end of the extension with the balloon uninflated.

FIG. 4 is a diagrammatic view of sections of a hypotube portion of an embodiment of a guide extension catheter.

FIG. 5 is flow diagram of a method of delivering a medical device using an embodiment of the guide extension catheter.

### DETAILED DESCRIPTION OF EMBODIMENTS

As used herein, a “guide catheter” or “guiding catheter” refers to a type of catheter that acts as a conduit for supporting device advancement through relatively larger vessels within a subject's vasculature, e.g., arteries or coronary ostium, but which is not generally suited for reaching into deeply intubate branch vessels.

As used herein, a “guide extension catheter” refers to a type of catheter that is advanced within a guiding catheter and into arteries and branch vessels, and which provides additional back-up support for advancing a guide wire through tortuous, angulated and calcified vessels to deliver treatment, e.g., a stent or balloon to a target location within a vessel.

As used herein, a “guide wire” or “cardiac wire” refers to a wire that is advanced through a guiding catheter to deliver a device for treatment at a target location.

According to embodiments of the invention, a guide support 100 is configured to be used in combination with conventional guide catheters and guide extension catheters to enhance the stability of such catheters during interventional procedures for treatment of obstructions within vasculature. The improved stability is provided by positioning a balloon at the distal end of the guide extension catheter that can be inflated against the inner surface of a blood vessel to anchor the guide extension catheter in place, thereby providing additional support while advancing equipment, such as stents, coronary balloons, and microcatheters to be transported towards areas of need. When the guide extension catheter is no longer needed, the balloon is deflated and the catheter can be withdrawn.

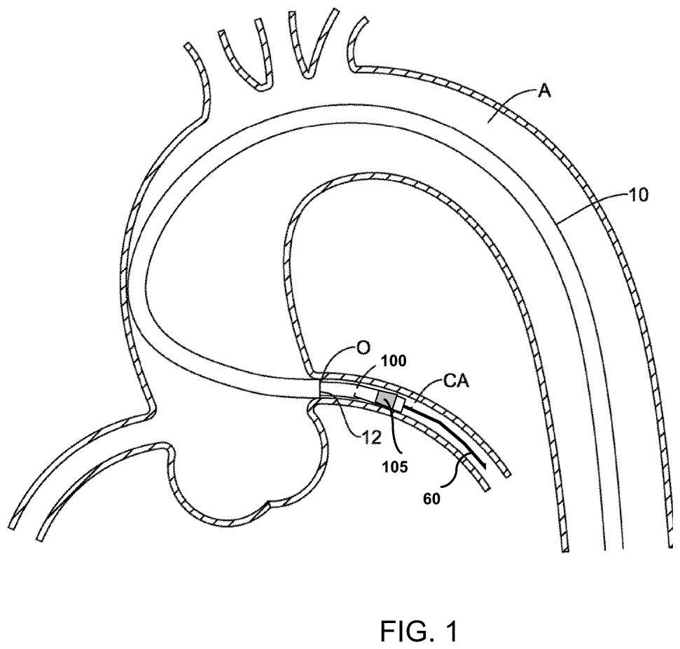

FIG. 1 illustrates an exemplary deployment of an embodiment of the inventive guide extension device 100 in conjunction with a cardiac procedure. The guide extension catheter 100 extends through a guide catheter 10 and beyond its distal end 12 into the coronary artery (CA). Guide extension catheter 100 extends beyond distal end 12 of guide catheter 10 to support guide wire 60, which delivers the treatment, e.g., a stent or balloon. Guide extension catheter 14 extends beyond the ostium (O) of the coronary artery CA and into a portion of the coronary artery CA. By extending beyond the ostium O, the extension catheter 100 can serve to stabilize the positioning of guide catheter 10 and allow for improved access to the coronary artery CA for a number of cardiac interventions. This illustration is provided for reference only. As will be recognized by those of skill in the art, guide catheters, guide wires, and their uses in a variety of different interventional and diagnostic procedures are well known in the art and, therefore, will not be described herein in further detail.

In general, a catheter assembly preferably has two distinct features. First, the catheter assembly must have sufficient “pushability” or axial strength to enable a longitudinal force to be transmitted through the assembly so that the physician can push the catheter assembly through the vascular system to the stenosis. The catheter assembly should also be sufficiently flexible so that the catheter assembly has good “trackability” so as to enable the physician to navigate the tortuous passages of the patient's vascular system.

To satisfy these criteria, catheter assemblies are often formed with a stiff proximal end, i.e., a pushing portion, and a more flexible distal end, i.e., a tracking portion. A hypotube formed of a metallic material such as stainless steel is often used at the proximal section, while the distal section of the assembly is often manufactured from a more flexible, polymer material. Thus, the hypotube is relatively stiff, enabling the assembly to have good pushability while the distal end is more flexible, providing the assembly with sufficient trackability.

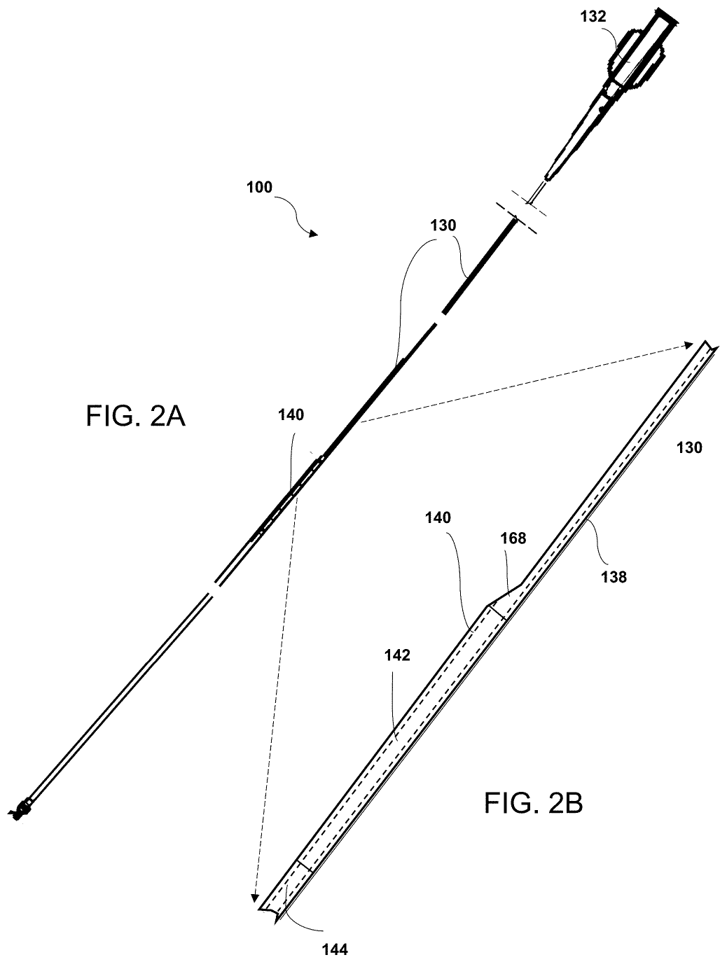

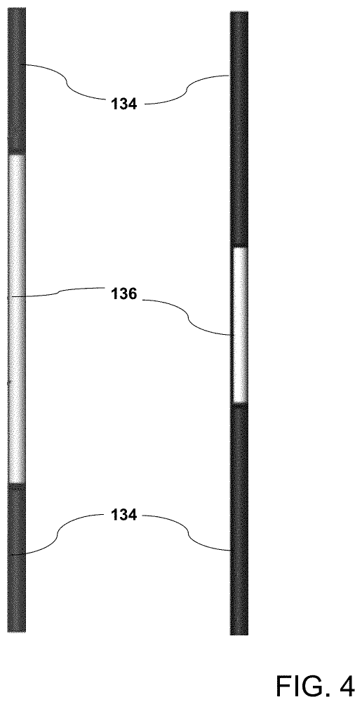

Referring to FIG. 2A, guide extension 100 includes a hypotube 130, which defines the proximal section of the assembly that connects to a conventional Luer connector 132 or similar connector through which an inflation fluid may be injected. Connector 132 preferably includes strain relief. Hypotube 130 is a small diameter, thin-walled elongated tube with sufficient strength and stiffness to provide the desired manipulability without kinking. In an exemplary embodiment, hypotube 130 is formed from 316 stainless steel with an inner diameter of around 0.38 mm-0.41 mm (˜0.016″) and an outer diameter of around 0.48 mm-0.50 mm (0.019″-0.020″). The length of hypotube 130 will depend on the type of procedure. In an exemplary assembly for performing a PCI procedure with access via a patient's wrist or groin, the length of hypotube 130 may be around 120-125 cm (˜48″). This sample length should not be considered limiting. In some embodiments, modifications in the stiffness and/or surface friction of the hypotube may be made along the entire length or at different sections along the tube length by applying a coating to the outer surface. In an exemplary implementation, a thin (˜0.2-3 μm; 0.001″) anti-friction and/or hydrophilic coating, e.g., Teflon® (PTFE) may be applied along the entire outer surface or leaving a few short sections of a several centimeters (inches) each with no coating. These variations in the coating of the hypotube may be used as positioning markers as well as facilitating handling of the assembly. FIG. 4 provides illustrative examples of different sections of hypotube 130 that are PTFE coated 134 and uncoated 136. Uncoated sections 136 of about 25 cm (˜10″) and 12.5 cm (˜5″) in width may be positioned at, for example, about 50 cm (˜20″) and about 85 cm (˜34″), respectively, from the proximal end of hypotube 130.

Referring to FIG. 2B, hypotube 130 is joined to the proximal end of guide extension 140. Wire entry port 168 provides an opening through which a guide wire (not shown) may be inserted into the extension. In some embodiments, the wire entry port 168 may optionally have a radio-opaque marker band to serve as an indicator for guiding the guide wire and device entry into the extension. Extension 140 includes a polymer (e.g., PEBAX®) tube 145 that may be reinforced using braided or coiled metal, plastic, graphite, or composite structures. In the exemplary embodiment, the polymer tube 145 has an inner diameter of about 1.5 mm-1.6 mm (0.062″). The coiled reinforcement is formed using 316 SS wire, 0.05 mm×0.025 mm with a 0.75 mm pitch. Extension 140 may be lined on its interior surface with an anti-friction PTFE liner 146. The reinforcement coil is coated on its exterior with a PEBAX® jacket 148, resulting in an outer diameter of about 1.9 mm-2.0 mm (0.078″). Note that while PEBAX® is commonly used in guide catheters and guide extension catheters, alternative materials may also be used. In some embodiments, different PEBAX® material laminations may be applied to make portions of the extension more or less stiff. In the illustrated example, proximal section 142 of extension 140 has a PEBAX® 72D coating to impart greater stiffness along a short section of the extension to enhance steerability. In the exemplary embodiment, proximal section 142 is approximately 25 mm (˜1.0″). The remaining length of extension 140, i.e., distal section 144, may be coated (laminated) with PEBAX® 42D layer to make this section softer with greater flexibility. Extension 140 may have a length of approximately 20 to 30 cm (˜9″-12″).

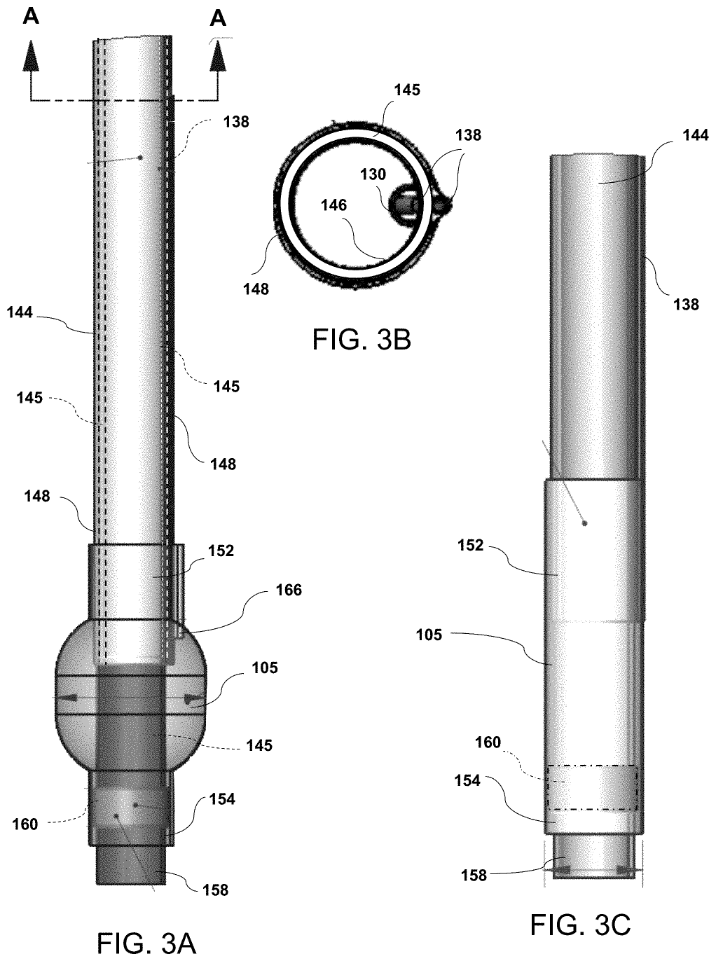

Extension 140 may be secured to hypotube 130 using conventional methods known in the for example, welding or bonding. An inflation lumen 138 formed from polyamide or similar polymer runs through the entire length of hypotube 130 and along a portion of extension 140 as will be discussed in more detail with reference to FIGS. 3A-3C.

FIGS. 3A-3C illustrate the distal end 144 of extension 140. Inflation lumen 138, which extends the working length of the catheter 100, running through hypotube 130 and is affixed to tube 145 by the lamination provided by PEBAX® jacket 148. FIG. 3A illustrates an inflated balloon while FIG. 3C shows the balloon uninflated. The cross-section provided in FIG. 3B shows the positioning of lumen 138, which is retained within the interior of hypotube 130 until it connects to extension 140, passing radially through a skived opening in tube 145 to be secured against the outer surface of tube 145 by the PEBAX® jacket 148. Lumen 138 continues toward the distal end of extension 140, terminating inside balloon 105. In an exemplary embodiment, balloon 105 is an annular sleeve formed from a thermoplastic polyurethane (TPU) or similar elastomer with high durability and flexibility. Such balloons, commonly referred to as “POBA” or “percutaneous old balloon angioplasty” balloons, are well known in the art.

In some embodiments, the balloon may be a non-compliant, semi-compliant, or highly compliant balloon formed from one or more materials such as polyurethane, polyolefin copolymer, latex, nylon, PEBA (polyether block amide), or other material. Use of compliant elastomeric materials allows the balloon to be soft (i.e., easily deformable and/or conformable to an area of targeted tissue) when fully inflated. Non-compliant material allows the balloon to be inflated to high pressures without deformability.

Balloon 105 has a proximal annular tail (or edge) 152 and a distal annular tail (edge) 154, each of which is sealed tightly over the extension tube. The airtight seal between the inner surface of edge 152 and the outer surface of PEBAX® jacket 148 may be achieved using an appropriate biocompatible adhesive, chemical bonding, or laser welding. Such sealing procedures are well known in the art. The seal at edge 152 secures inflation lumen 138 so that the opening at its distal end exits into the interior of expandable section 156 of balloon 105. Inflation fluid introduced into lumen 138 at its proximal end causes the balloon to expand radially to a maximum diameter of approximately 4 mm. The volume of fluid introduced will determine the degree of inflation. Once inflated, the balloon stabilizes the distal end of the guide extension within the vessel, which centers the guide wire coaxially within the vessel opening, allowing it to be advanced past an obstruction.

Tail 154 of balloon 105 is sealed to the outer surface of tube 145 using the attachment/sealing methods previously described. A marker band 160 formed of a radiopaque material such as platinum/iridium alloy may be sandwiched between the outer surface of tube 145 and the inner surface of tail 154. The marker band 160 may be deposited, applied as a thin film paste or as a thin foil, or other application method known in the art. The distal end 158 of tube 145 preferably extends only a short distance beyond the edge of tail 154 so that the balloon is located as close as possible to the distal end of the extension. The edges of distal end 158 may be chamfered or rounded.

The outer surface of balloon 105 may optionally be coated with, or the balloon material may be impregnated with, one or more substances to aid in the treatment or in the manipulation of the device. For example, a pharmacological agent, i.e., drug or medication, may be incorporated into the balloon surface for application at the site of the stenosis. Upon inflation, cells or micropores within the balloon's surface expand, releasing their payload to the surrounding vessel walls. The pharmacological agent may include an additive to enhance absorption of the drug into the vascular wall. In another embodiment, one or more drug or a combination of drug(s) and excipients may be contained within a polymer coating that is applied to the balloon to allow the drug to diffuse into the vessel wall when contacted by the expanded balloon.

Expansion of the balloon may be effected by injecting a fluid through the inflation lumen, which is connected at its proximal end to a Luer connector 132 or similar connector. Non-limiting examples of appropriate fluids include iodinated contrast solutions, saline solutions, sterile water, and air. The use of a syringe allows injection of a precisely measured volume of fluid appropriate for the balloon size into the connector by applying gradual and constant force. Alternatively, a small pump may be used to inject and withdraw the fluid from the balloon.

In some applications, inflation of the balloon may be used to decrease blood flow into at least one of arterial dissection planes and subintimal space. This assists with keeping blood within the blood vessel in which the guide extension catheter is advancing.

To perform a procedure using the inventive guide extension support, the coronary wire is used to guide the guide extension catheter to the point of either abutting a lesion or until the guide extension catheter can no longer be advanced forward. The wire may be an elongated solid wire of constant or varying dimensions and can be made of a polymeric or metallic material, such as high tensile stainless steel (e.g., 304V, 304L or 316LV), mild steel, nickel-titanium alloys, nickel-chromium-molybdenum alloys, nickel-copper alloys, nickel-tungsten alloys or tungsten alloys. At the point that the wire can no longer be advanced, the balloon is inflated to apply a substantially uniform radial outward force against the walls of the vessel. With the distal end of the guide extension stabilized, the guide wire is further advanced to the target at which a device such as a stent, a balloon, a microcatheter, or a combination thereof is to be deployed. After treatment, the inflation fluid is withdrawn from the balloon to deflate it, e.g., by retracting a syringe, and the assembly is withdrawn.

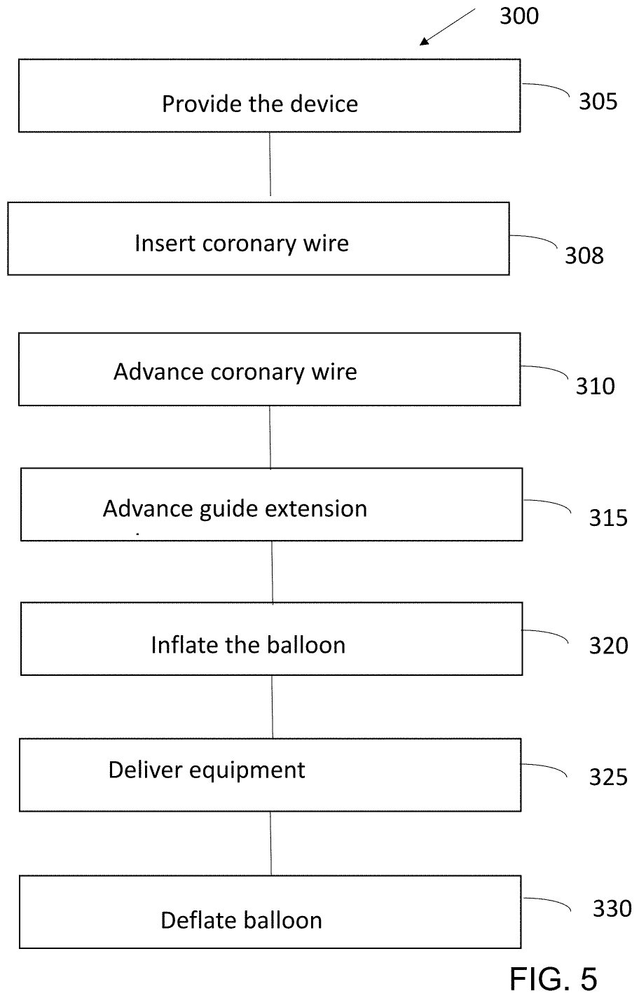

FIG. 5 provides a simple flow diagram of steps of an exemplary PCI procedure 300 using the improved guide support described herein. In typical PCI procedures, a diagnostic catheter will have been used to identify the location of the stenosis. The diagnostic catheter is removed and exchanged for a guide catheter. The guide extension catheter with improved guide support is provided in preparation (step 305). After the guide catheter is placed in the ostium of the respective artery, a guide wire is inserted (step 308) and advanced through the catheter toward the lesion (step 310). Over the wire, the guide extension catheter is advanced through the guide catheter and then into the vessel (step 315). Guide extension catheter is advanced until it reaches a stenotic lesion or until it can no longer be advanced. At this point, the support balloon near the distal end of the extension is inflated to press against the inner surface of the blood vessel (step 320), anchoring the guide extension catheter in place and/or occluding blood flow. Over the guide wire within the guide extension catheter, additional equipment is delivered (step 325) as needed to perform the intervention (such as balloons, stents, or microcatheters). After treatment, e.g., placement of the stent or expansion/deflation of the treatment balloon, the support balloon is deflated (step 330) and the guide extension catheter is withdrawn. The guide support system may be used in coronary procedures as well as other peripheral vascular procedures involving blood vessels, arteries, arterioles, and capillaries.

The overall guide extension catheter configuration described herein is intended to be illustrative. Existing guide extension catheters may employ variations in materials and designs, for example, solid pushwires, different coiled or braided extensions, and tapered ends. The enhanced stability provided by disposing an inflatable balloon a short distance from the distal end of the extension to anchor the end of the extension within the vessel would equally benefit such other designs. Accordingly, the improvement is not limited to the specific illustrated guide extension catheter configuration.

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

1. A guide extension catheter with improved stability comprising:
an elongated pushing portion configured for guiding a guide wire into a vessel, the pushing portion having a proximal end and a distal end;
an extension portion disposed at the distal end of the pushing portion, the extension portion comprising a tube having a proximal portion, a distal portion, and an interior configured for slidably receiving a guide wire configured for delivery of a treatment device to a treatment target;
a balloon disposed near a distal end of the extension portion, the balloon having an annular configuration with an expandable center portion and edges on either side of the center portion, the edges sealed to an outer surface of the extension portion to define a fluid-tight seal;
an inflation lumen extending along a length of the pushing portion and the extension portion, wherein a distal end of the inflation lumen feeds into an interior of the balloon;
a fluid connecter connected to a proximal end of the inflation lumen, the fluid connector configured for introducing fluid into the inflation lumen to expand the balloon;
wherein introduction of fluid into the balloon causes the balloon to expand radially outward from the extension to apply pressure against an inner wall of the vessel to anchor the distal end of the extension within the vessel.

2. The guide extension catheter of claim 1, wherein the elongated pushing portion comprises a hypotube formed of a metal material, and wherein the inflation lumen is retained within an interior of the hypotube.

3. The guide extension catheter of claim 2, wherein an exterior surface of the elongated pushing portion is at least partially coated with a low-friction material.

4. The guide extension catheter of claim 1, wherein the tube of the extension portion comprises a flexible polymer material having an inner surface coated with a low-friction material.

5. The guide extension catheter of claim 4, wherein at least a portion of a length of the tube is reinforced with a coiled wire.

6. The guide extension catheter of claim 4, wherein the tube is formed of polymer materials having different stiffnesses, wherein the proximal portion of the extension has greater stiffness than the distal portion.

7. The guide extension catheter of claim 1, wherein the extension portion further comprises a polymer jacket disposed on an outer surface of the tube, wherein the polymer jacket secures the inflation lumen against the outer surface of the tube.

8. The guide extension catheter of claim 1, further comprising a radio-opaque marker formed on an outer surface of the tube, sandwiched beneath a distal-most edge of the balloon.

9. An improved guide extension catheter comprising an elongated pushing portion having a distal end connected to a tubular extension portion, the improvement comprising:
a balloon disposed near a distal end of the extension portion, the balloon having an annular configuration with an expandable center portion and edges on either side of the center portion, the edges sealed to an outer surface of the extension portion to define a fluid-tight seal;
an inflation lumen extending along a length of the pushing portion and the extension portion, wherein a distal end of the inflation lumen feeds into an interior of the balloon; and
a fluid connecter connected to a proximal end of the inflation lumen, the fluid connector configured for introducing fluid into the inflation lumen to expand the balloon;
wherein introduction of fluid into the balloon causes the balloon to expand radially outward from the extension to apply pressure against an inner wall of the vessel to anchor the distal end of the extension within the vessel.

10. The guide extension catheter of claim 9, wherein the elongated pushing portion comprises a hypotube formed of a metal material, and wherein the inflation lumen is retained within an interior of the hypotube.

11. The guide extension catheter of claim 10, wherein an exterior surface of the elongated pushing portion is at least partially coated with a low-friction material.

12. The guide extension catheter of claim 9, wherein the extension portion comprises a tube formed from a flexible polymer material having an inner surface coated with a low-friction material.

13. The guide extension catheter of claim 12, wherein at least a portion of a length of the tube is reinforced with a coiled wire.

14. The guide extension catheter of claim 12, wherein the tube is formed of polymer materials having different stiffnesses, wherein a proximal portion of the extension has greater stiffness than the distal portion.

15. The guide extension catheter of claim 12, wherein the extension portion further comprises a polymer jacket disposed on an outer surface of the tube, wherein the polymer jacket secures the inflation lumen against the outer surface of the tube.

16. The guide extension catheter of claim 9, further comprising a radio-opaque marker disposed on an outer surface of the tube sandwiched beneath a distal-most edge of the balloon.

17. An improved method for delivering a treatment device to a target location within peripheral vasculature of a subject, the method comprising advancing a guide wire toward a target location within the peripheral vasculature using a guide catheter, the improvement comprising:
providing a guide extension catheter having an elongated pushing portion having a distal end connected to a tubular extension portion configured for slidably receiving the guide wire, wherein the extension portion has a balloon disposed near a distal end of the extension portion, the balloon having an annular configuration with an expandable center portion with sealed edges on either side of the center portion defining a fluid-tight seal, and an inflation lumen extending along a length of the pushing portion and the extension portion, wherein a distal end of the inflation lumen feeds into an interior of the balloon;
advancing the guide extension catheter and the guide wire through the guide catheter and into the subject's peripheral vasculature toward the target location until resistance is encountered;
stabilizing a distal end of the guide extension catheter within the peripheral vasculature by injecting a fluid into a proximal end of the inflation lumen and into the balloon to expand the balloon to apply pressure against an inner surface of the peripheral vasculature;
advancing a distal end of the guide wire beyond the distal end of the guide extension catheter to the target location;
delivering the treatment device to the target location;
retracting the fluid from the balloon; and
withdrawing the guide extension catheter, guide wire, and guide catheter from the subject.

18. The improved method of claim 17, wherein the elongated pushing portion comprises a hypotube formed of a metal material, and wherein the inflation lumen is retained within an interior of the hypotube.

19. The improved method of claim 18, wherein an exterior surface of the elongated pushing portion is at least partially coated with a low-friction material.

20. The improved method of claim 17, wherein the extension portion comprises a tube formed from a flexible polymer material having an inner surface coated with a low-friction material.

21. The improved method of claim 20, wherein at least a portion of a length of the tube is reinforced with a coiled wire.

22. The improved method of claim 20, wherein the tube is formed of polymer materials having different stiffnesses, wherein a proximal portion of the extension has greater stiffness than the distal portion.

23. The improved method of claim 20, wherein the extension portion further comprises a polymer jacket disposed on an outer surface of the tube, wherein the polymer jacket secures the inflation lumen against the outer surface of the tube.

24. The improved method of claim 17, further comprising a radio-opaque marker disposed on an outer surface of the tube sandwiched beneath a distal-most edge of the balloon.

25. The improved method of claim 17, where the treatment device is at least one of a stent, a coronary balloon, and a microcatheter.
